(19) United States
(12) Patent Application Publication
(10) Pub. No.: US 2015/0309134 A1
(43) Pub. Date: Oct. 29, 2015

(54) DIFFUSION-INSENSTIVE VELOCITY SELECTIVE LABELLING MODULE FOR MAGNETIC RESONANCE IMAGING

(71) Applicant: ISIS INNOVATION LTD, Oxford (GB)

(72) Inventors: James MEAKIN, Oxford (GB); Peter JEZZARD, Oxford (GB)

(21) Appl. No.: 14/660,576

(22) Filed: Mar. 17, 2015

Related U.S. Application Data
(60) Provisional application No. 61/955,132, filed on Mar. 18, 2014.

(51) Int. Cl. G01R 33/48; A61B 5/026

(52) U.S. Cl. CPC G01R 33/48; A61B 5/0263

(57) ABSTRACT

A velocity selective preparation method, for Velocity Selective Magnetisation Transfer Insensitive labelling technique (VS-TILT), said VS-TILT method using non-selective RadioFrequency (RF) pulses and magnetic field gradients to modulate the longitudinal magnetization of moving spins in magnetic resonance imaging that is insensitive to diffusion effects, said method comprising the steps of: a) play out two velocity selective pulses: VS-A and VS-B, sequentially without any spoiling between said pulses; b) each individual pulse VS-A and VS-B having half the first gradient moment m₁ of the original velocity selective pulse; c) assigning the VS-TILT tag condition gradients to have the same polarity, such that total m₁ is perserved; and d) assigning the VS_TILT control condition, negating the n gradients in the first pulse such total m₁=0, but the b-value remains unchanged.

### RELATED APPLICATIONS

This application claims the benefit of U.S. Provisional Patent Application Ser. No. 61/955,132 filed on Mar. 18, 2014, the disclosure of which is hereby incorporated by reference.

### TECHNICAL FIELD

The present invention generally relates to measuring blood perfusion and more particularly, relates to a method to modulate the longitudinal magnetization of moving spins in magnetic resonance imaging that is insensitive to diffusion effects. The method of the present invention generates contrast based on differences in velocity including perfusion imaging, angiography and venography, to name but a few examples.

### BACKGROUND

Arterial Spin Labeling (ASL) uses endogenous blood water as a freely diffusible tracer to noninvasively quantify perfusion. Classical techniques including pulsed and continuous ASL invert spins upstream to the imaging volume and then image spins that have exchanged into tissue. The necessary spatial separation between tagging and imaging regions can result in long bolus arrival times, which is one of the largest sources of error in the quantification of ASL. This is especially problematic in situations where bolus arrival time is already increased, such as stroke, white matter or skeletal muscle, leading to decreased signal-to-noise ratio or erroneous perfusion values.

Velocity-Selective ASL (VSASL) is a variant of pulsed ASL that eliminates the bolus arrival time by labelling the blood much closer to the tissue bed. VSASL uses non-selective radiofrequency (RF) pulses and magnetic field gradients to modulate the longitudinal magnetization of the spins (M_{z}) as a function of their velocity. The velocity-selective (VS) preparation saturates spins above a certain cut-off velocity (V_{c}), which are then imaged after they have exchanged into tissue. Through setting V_{c} to a value corresponding to the blood velocity at the arteriole-capillary bed interface the technique is made insensitive to bolus arrival time, as the tag is being generated within the imaging volume itself.

Several artefacts hinder accurate quantification of VSASL. B₁ and B₀ inhomogeneities lead to an underestimation of perfusion due to spatial variations in tagging efficiency. Additionally, the VSASL sequence is not eddy current balanced as the VS gradients played out in the tag acquisition are not played out in the control acquisition.

Furthermore, other artifacts, such as diffusion effects, play a role in quantification of VSASL. Diffusion effects can be split into two different categories—bulk motion and diffusion.

Accordingly there is a need to address the aforementioned deficiencies. The aim of the present invention is therefore to provide a method that overcomes the deficiencies named above. The present invention is a method to modulate the longitudinal magnetisation of moving spins in MRI that is insensitive to the diffusion effects mentioned above.

### SUMMARY

In an embodiment there is provided a velocity selective preparation method, for Velocity Selective Magnetisation Transfer Insensitive labelling technique (VS-TILT), said VS-TILT method using non-selective RadioFrequency (RF) pulses and magnetic field gradients to modulate the longitudinal magnetization of moving spins in magnetic resonance imaging that is insensitive to diffusion effects, said method comprising the steps of:

a) play out two velocity selective pulses: VS-A and VS-B, sequentially without any spoiling between said pulses;

b) each individual pulse VS-A and VS-B having half the first gradient moment ml of the original velocity selective pulse;

c) assigning the VS-TILT tag condition gradients to have the same polarity, such that total ml is perserved;

d) assigning the VS_TILT control condition, negating the n gradients in the first pulse such total m1=0, but the b-value remains unchanged.

In the tag condition, two +90° RF pulses may be played out to produce a spatially selective 180° inversion.

In the control condition a +90°-90° pattern may be played out, to balance magnetization transfer effects.

A B1 Insensitive Rotation pulse of order 4 may be used as the base velocity selective pulse.

A B1 Insensitive Rotation pulse of order 8 may be used as the base velocity selective pulse.

A B1 Insensitive Rotation pulse of order 16 may be used as the base velocity selective pulse.

A B1 Insensitive Rotation pulse of order 32 may be used as the base velocity selective pulse.

The cut off velocity may be set in the range 2-16 cm/s.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 depicts the VSASL pulse sequence that is used in the method of the present invention.

FIGS. 2A to 2C illustrate the perfusion overestimation due to tissue diffusion when using B1 insensitive rotation with an order of 8, BIR-8 VSASL for Cerebrospinal Fluid (CSF), grey matter and white matter.

FIGS. 3A to 3F illustrate the perfusion overestimation due to tissue motion for CSF, grey matter and white matter for v_{cut}=2 cm/s and 8 cm/s.

FIGS. 4A to 4C show a BIR-4 VS-TILT pulse diagram showing normalized RF power, RF phase and velocity selective gradient, for BIR-4 tag condition, control condition negating frequency sweep and control condition negating gradients.

FIGS. 5A to 5C show a BIR-8 VS-TILT pulse diagram showing normalized RF power, RF phase and velocity selective gradient, for BIR-8 tag condition, control condition negating frequency sweep and control condition negating gradients.

FIGS. 6A to 6C show a BIR-4 VS-TILT off-resonance response for moving spins for the tag condition, frequency sweep negation control condition and gradient polarity negation.

FIGS. 7A to 7C show a BIR-8 VS-TILT off-resonance response for moving spins for the tag condition, frequency sweep negation control condition and gradient polarity negation

FIGS. 8A to 8E show the M_{Z} of static spins after a BIR-4 VS-TILT preparation in the presence of eddy currents with time constants τ as a function of distance from isocentre, for the tag condition (8A), the control condition when negating the frequency sweep of VS-A (8B), the control condition when negating the gradient polarity of VS-A (8C), ΔM subtraction when negating frequency sweep (8D) and ΔM subtraction when negating gradients (8E).

FIGS. 9A to 9E show the M_{Z} of static spins after a BIR-8 VS-TILT preparation in the presence of eddy currents, for the tag condition (9A), the control condition when negating the frequency sweep of VS-A (9B), the control condition when negating the gradient polarity of VS-A (9C), ΔM subtraction when negating frequency sweep (9D) and ΔM subtraction when negating gradients (9E).

FIGS. 10A to 10G shows the ΔM subtraction errors in a static phantom.

FIGS. 11A to 11E illustrate the saturation efficiency in flowing tap water using BIR-8 as the velocity selective pulse for both the VSASL and VS-TILT labeling schemes.

FIGS. 12A to 12C depict the velocity selective preparations used in the in vivo data for V_{cut}=2 cm/s for VSASL, VSASL-II and VS-TILT

FIGS. 13A to 13D show perfusion maps in a slice containing the ventricles for all subjects for VSASL (13A), VSASL-II (13B) and VS-TILT (13C), and the mean grey matter f across all subjects±SD (13D).

FIGS. 14A to 14I show VIM, VASO-IVIM and FLAIR-IVIM in vivo.

FIGS. 15A and 15B show the Theoretical M_{z} after the application of a cosinusoidal velocity selective pulse for vessels with plug and laminar velocity distributions, when considering an anisotropic (15A) or isotropic (15B) vessel distribution.

FIGS. 16A and 16B show the theoretical bolus shapes for laminar flowing vessel networks. The response to a velocity selective pulse of an anisotropic distribution, or a single vessel, that is aligned with the velocity selective gradient where 8=0 (16A) and the response of an isotropic distribution of laminar vessels (16B).

### DETAILED DESCRIPTION

Having summarized various aspects of the present disclosure, reference will now be made in detail to the description of the disclosure as illustrated in the drawings. While the disclosure will be described in connection with these drawings, there is no intent to limit it to the embodiment or embodiments disclosed herein. On the contrary, the intent is to cover all alternatives, modifications and equivalents included within the spirit and scope of the disclosure as defined by the appended claims.

Physiological noise due to fluctuations of the large fraction of static tissue in the acquired voxel reduces the sensitivity of ASL data. These fluctuations can be reduced with background suppression and/or cardiac RETROICOR. In the present invention, the additional physiological effects during the velocity selective pulse itself are considered. These could generate additional systematic errors beyond the physiological noise in spatially labeled ASL, since in VSASL the labeling pulse is applied to the entire RF field of view. Here, only the velocity selective pulse is considered as only this is modified between tag and control, potentially resulting in a systematic bias in the perfusion weighted subtraction. All other pulses and crushers are identical between tag and control, so will not lead to a systematic bias.

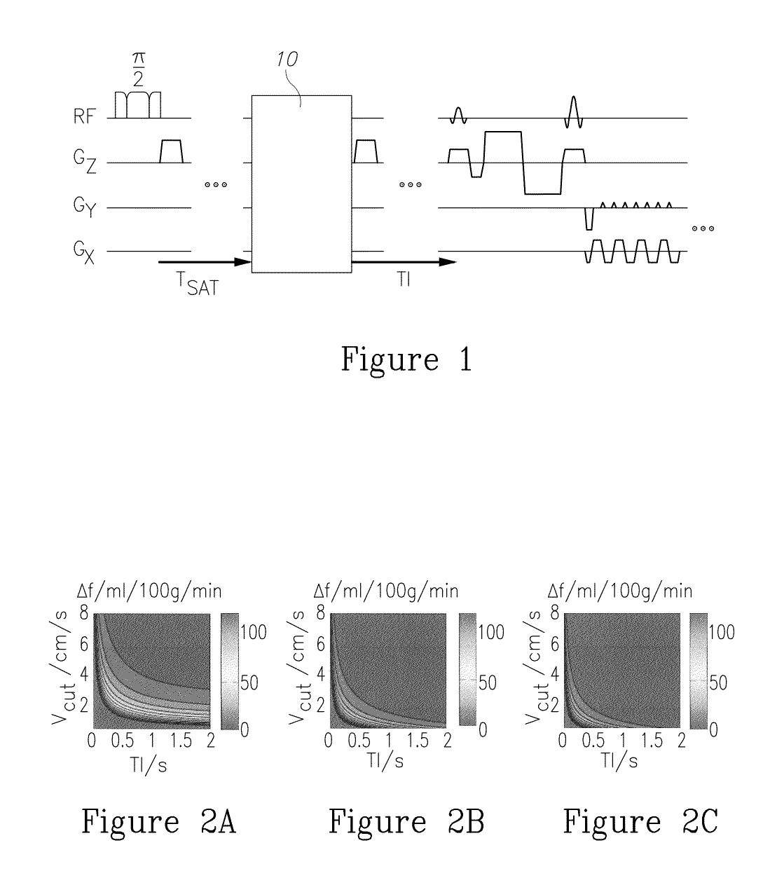

Consider a voxel that has tissue with equilibrium magnetisation M_{0,t} and relaxation times T_{1,t} and T_{2,t}. At the start of the VSASL pulse sequence the magnetisation is reset by the pre-saturation pulse, which is shown in FIG. 1. The VSASL pulse sequence used herein consists of three sequence building blocks. First there is a global pre-saturation using a non-selective π/2 BIR-4 pulse to remove any spin history effects. This is followed by a saturation delay T_{SAT} and the velocity selective preparation in either the tag or control condition as shown at 10. This is followed by the inflow time delay, TI, and a spin echo EPI readout for each slice. The crusher gradients have VENC=V_{cut}in both tag and control conditions, and is applied on the same axis as the velocity encoding gradients.

At the time of the velocity selective pulse (T_{SAT}) the magnetisation is given by

$${M}_{t} ( {T}_{SAT} ) = {M}_{0 , t} \cdot [ 1 - exp ( - \frac{{T}_{SAT}}{{T}_{1 , t}} ) ] \qquad ( 1.1 )$$

The velocity selective pulse with saturation efficiency is then applied. If there are any differences in the velocity selective pulse between tag and control the difference in tissue M_{Z}, will be given by:

$$Δ {M}_{t} ( {T}_{SAT} ) = {M}_{0 , t} \cdot [ 1 - exp ( - \frac{{T}_{SAT}}{{T}_{1 , t}} ) ] \cdot α \cdot ( 1 - Δβ ) \qquad ( 1.2 )$$

where Δβ is a factor that accounts for these perturbations. Ideally, the background tissue is not perturbed by the velocity selective pulse and Δβ=1. However, in VSASL the contrast is fundamentally generated by a difference in first gradient moment, m₁, of the velocity selective pulse. For the tag condition

$${m}_{1} = \frac{π}{γ {V}_{cut}}$$

and for the control m₁=0. This results in a cosinusoidal variation with velocity in M_{Z}, in the tag condition, but M_{Z} is constant for all velocities in the control condition. For this difference in m₁, Δβ given by

$$Δ {β}_{v} = cos ( γ \cdot {m}_{1} \cdot v ) = cos ( π \cdot \frac{{v}_{t}}{{V}_{cut}} ) \qquad ( 1.3 )$$

where υₜ is the velocity of the tissue projected along the direction of the velocity selective gradient.

In current VSASL implementations the difference in m₁ is achieved by playing out the velocity selective gradients in the tag condition but turning them off in the control condition. Along with a difference in m₁ between tag and control there is also a difference in the diffusion b-value. For diffusion effects, the Δβ term is

Δβ_{D}=exp(−b(V_{cut})·ADCₜ) (1.4)

where ADCₜ is the apparent diffusion coefficient of the tissue and b(V_{cut}) is the difference in b-value for the chosen V_{cut}.

The difference in tissue M_{Z} at the time of the acquisition is then given by

$$Δ {M}_{t} ( T 1 ) = {M}_{0 , t} \cdot [ 1 - exp ( - \frac{{T}_{SAT}}{{T}_{1 , t}} ) ] \cdot α \cdot ( 1 - Δβ ) \cdot exp ( - \frac{T 1}{{T}_{1 , t}} ) \cdot exp ( - \frac{TE}{{T}_{2 , t}} ) \qquad ( 1.5 )$$

where TI is the inflow time and TE is the echo time. Equation 1.5 above shows that the difference in m₁ and b lead to a positive contribution from tissue in the perfusion weighted subtraction as Δβ<+1 in both cases.

The bolus arrival time in VSASL is assumed to be zero, so the standard PASL model describes the contribution from blood as

$$Δ {M}_{b} ( TI ) = {M}_{0 , B} \cdot [ 1 - exp ( - \frac{{T}_{SAT}}{{T}_{1 , B}} ) ] \cdot α \cdot f \cdot TI \cdot {q}_{p} ( f ) … exp ( - \frac{TI}{{T}_{1 , B}} ) \cdot exp ( - \frac{TE}{{T}_{2 , B}} ) \qquad ( 1.6 )$$

where the B subscripts indicate blood, f is perfusion and qₚ(f) takes into account the difference in relaxation rates between blood and tissue.

Consider a voxel where the true perfusion is zero and the only signal in the VSASL experiment is from tissue. If perfusion were to be quantified using equation 1.6, the erroneous perfusion signal from tissue, Δf, would be given by equating equations 1.6 and 1.5:

ΔMₜ(TI)=ΔM_{B}(TI) (1.7)

such that:

$$Δ f = \frac{{M}_{0 , t} \cdot [ 1 - exp ( - \frac{{T}_{SAT}}{{T}_{1 , i}} ) ] \cdot ( 1 - Δ β ) \cdot exp ( - \frac{TI}{{T}_{1 , i}} ) \cdot exp ( - \frac{TE}{{T}_{2 , i}} )}{{M}_{0 , B} \cdot [ 1 - exp ( - \frac{{T}_{SAT}}{{T}_{1 , B}} ) ] \cdot TI \cdot {q}_{p} ( f ) \cdot exp ( - \frac{TI}{{T}_{1 , B}} ) \cdot exp ( - \frac{TE}{{T}_{2 , B}} )} \qquad ( 1.8 )$$

The systematic perfusion overestimation (equation 1.8) was then simulated using MAT-LAB r2012a (The Mathworks, Natick, Mass., USA). Three example tissue types were considered: Grey Matter, White Matter and Cerebrospinal fluid (CSF) with tissue properties summarised in table 1 below:

| Tissue | T₁/s | T₂/s | ADC/mm²/s | M_{0,t}/M_{0,CSF} |
|---|---|---|---|---|
| CSF | 3.7^{A} | 1.30^{C} | 2.7 × 10^{3 F} | 1.00^{G} |
| Grey Matter | 1.1^{A} | 0.11^{D} | 0.9 × 10^{3 F} | 0.89^{G} |
| White Matter | 0.8^{A} | 0.08^{D} | 0.7 × 10^{3 F} | 0.73^{G} |
| Blood | 1.6^{B} | 0.15^{E} | — | 0.87^{G} |

A VSASL acquisition was simulated with T_{SAT}=3 s and TE=30 ms. The diffusion b-value was calculated for a BIR-8 VSASL preparation with maximum gradient strength 20 mT/m, rise time 0.5 ms, and V_{cut} from 0.5 cm/s to 8 cm/s in steps of 0.05 cm/s. The TI was varied from 1 ms to 2 s and Δf from equation 1.8 with Δβ from equation 1.4, was calculated for CSF, Grey Matter and White Matter.

The effect of tissue motion was simulated using equation 1.8, with Δβ from equation 1.3. This simulation is independent of the velocity selective pulse used, and only depends on the m₁ imparted in the tag condition relative to the control condition. The diffusion b-value for the tissue motion simulation was assumed to be zero. Parameters used were T_{SAT}=3 s, TE=30 ms, TI was varied from 1 ms to 3 s in steps of 1 ms and the velocity of the tissue varied from −2 mm/s to +2 mm/s in steps of 1 μm/s.

The perfusion overestimation values due to tissue diffusion in BIR-8 VSASL are displayed in FIG. 2, shown for CSF in FIG. 2A, grey matter in FIG. 2B and white matter in FIG. 2C. It can be see that the overestimation is highest for CSF, as CSF has a greater ADC than grey matter or white matter. The overestimation increases as V_{cut} decreases, due to the increasing b-value of the velocity selective pulse. The error reduces as TI increases, as the tissue signal will decay with T_{1,t}. For TI=1 s at V_{cut}=2 cm/s the overestimation in a voxel of CSF will be +32.0 ml/100 g/min, grey matter will be +6.4 ml/100 g/min and white matter will be +2.8 ml/100 g/min.

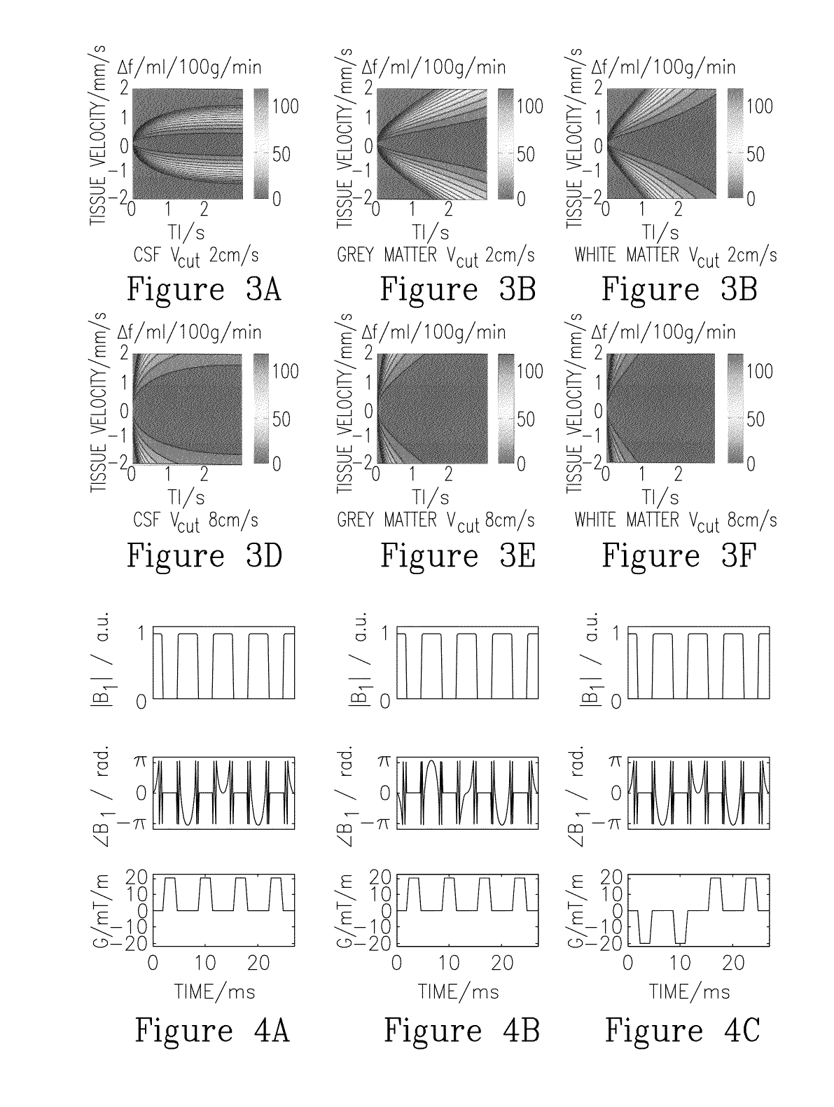

The results for the simulation of tissue motion at V_{cut}=2 cm/s and 8 cm/s are displayed in FIG. 3. The errors are again largest for CSF, due to the long T₁ of the tissue. At V_{cut}=2 cm/s and TI=1 s, CSF that has velocity of ±1 mm/s will cause an overestimation of perfusion of +93.8 ml/100 g/min, grey matter at ±1 mm/s will cause an overestimation of +53.7 ml/100 g/min and white matter at ±1 mm/s an overestimation of +31.8 ml/100 g/min. These overestimations are reduced when using V_{cut}=8 cm/s to +5.9 ml/100 g/min for CSF, +3.4 ml/100 g/min for grey matter and 2.0 ml/100 g/min for white matter, respectively.

Although the tissue velocity and ADC may be small, the errors are on the order of the expected in vivo perfusion signal, especially for tissue motion of ±1 mm/s at V_{cut}=2 cm/s. Whilst the diffusion error could be eliminated by creating a diffusion insensitive labeling scheme, the errors due to motion are a fundamental bi-product of the blood-tissue contrast generation in VSASL.

The derivations show that physiological noise due to bulk motion is rectified in VSASL, and will always contribute to a perfusion overestimation. Depending on the tissue type, even a small velocity of motion during the labeling pulse can cause artefacts of the order of the genuine perfusion signal. The error increases as V_{cut} is reduced or as short TI values are used. Due to the cosinusoidal variation in velocity produced in the tag condition both positive and negative tissue velocities will result in an overestimation of perfusion. As the signal compartment of the background tissue is large, only a small velocity during the pulse (for example, 1 mm/s results in a displacement of 20 μm during the BIR-8 pulse) will result in significant perfusion overestimation.

It is important to note that these systematic errors will not be reduced when using traditional background suppression approaches. In traditional background suppression two inversion pulses are applied during the inflow time, between the labeling pulse and the readout excitation. This reduces the magnetisation of the background tissue at the time of signal readout so that any fluctuations from physiological variation are minimised, but the difference between the tagged and controlled blood magnetisation is preserved. It follows that any magnetisation difference between tag and control states caused by the labeling pulse will be preserved when using traditional background suppression techniques. As the diffusion and bulk motion errors are being generated during the labeling pulse, they will not be reduced when using inversion pulses during the inflow time.

However, the background suppression principle can be used to minimise the bulk motion errors in VSASL. The difference is that the background suppression must be played out before the velocity selective pulse. This results in the magnetisation of the background tissue being ≈0 at the time of the velocity selective pulse, so that it cannot be erroneously tagged.

In these simulations, quantification using only a single TI is considered. Alternatively, it is possible to add equation 1.8 as another signal component when fitting multi-TI data. However, the approach taken herewith is to remove these artefacts at the time of acquisition rather than in post processing, since if the BAT in VSASL can be approximated as being zero only a single TI needs to be acquired.

Although these simulations using literature values of tissue properties suggest that diffusion will not make a significant erroneous contribution to the VSASL signal, eliminating these effects will increase the accuracy of the perfusion measurement by reducing the systematic error.

Above it was shown that diffusion effects can cause an overestimation of perfusion in VSASL. This is because the velocity selective preparation has a higher diffusion b-value in the tag condition than in the control condition, as the gradients that impart the velocity sensitivity in the tag condition are simply turned off for the control condition.

Velocity selective preparations have also been used to isolate the venous blood compartment to determine Oxygen Extraction Freaction (OEF) in Quantitative Imaging of Extraction of oxygen and Tissue Consumption (QUIXOTIC). However, it was found in the prior art that the measured venous T₂ in grey matter was longer than the value in the sagittal sinus, indicating that the venous T₂ in grey matter was not being measured correctly. Other prior art methods used a diffusion balanced velocity selective preparation (VSEAN) that eliminated the mis-match between the grey matter and sagittal sinus T₂. These prior art methods thought that long T₂ components such as CSF were contaminating the QUIXOTIC measurement as they were likely to contribute erroneous signal through the diffusion effects mentioned above.

The velocity selective labeling scheme used in VSEAN requires complete spoiling of signal half way through the preparation, and a phase projection step. The VSEAN preparation is not directly applicable to VSASL as the resulting longitudinal magnetisation is proportional to

$${sin}^{2} ( π \frac{v}{{V}_{cut}} ) .$$

For a comparison with current VSASL techniques

$${M}_{z} ∝ cos ( π \frac{v}{{V}_{cut}} )$$

is required.

Below, a new diffusion balanced velocity selective preparation is proposed that is able to produce

$${M}_{z} ∝ cos ( π \frac{v}{{V}_{cut}} )$$

and is referred to as VS-TILT. The VS-TILT preparation is evaluated for robustness to eddy currents and ΔB₀, and is applied in vivo to determine empirically the magnitude of diffusion contamination in VSASL.

In the VSASL tag condition the velocity selective preparation is applied with first gradient moment

$${m}_{1}^{tag} = \frac{π}{γ {V}_{cut}}$$

and in the control condition m₁^{ctrl}=0. In standard VSASL the gradients are turned off in the control condition, which results in a difference in diffusion b-value compared to the tag condition. To balance the diffusion effects, a preparation with the same magnitude gradient pattern is desired whilst still achieving the desired difference in m₁. This is achieved by playing out two velocity selective pulses, VS-A and VS-B, sequentially without any spoiling between the pulses. The total first gradient moment after the application of VS-A and VS-B is simply

m₁=m_{1,A}+m_{1,B} (1.9)

In the tag condition the velocity selective pulses are applied with

$${m}_{1 , A}^{tag} = {m}_{1 , B}^{tag} = \frac{π}{2 γ {V}_{cut}} \qquad ( 1.10 )$$

so that the total first gradient moment is

$${m}_{1}^{tag} = \frac{π}{γ {V}_{cut}}$$

In the control condition VS-A and VS-B are played out with one of the first gradient moments negated

$$- {m}_{1 , A}^{ctrl} = {m}_{1 , B}^{ctrl} = \frac{π}{2 γ {V}_{cut}} \qquad ( 1.11 )$$

so that the total first gradient moment is m_{1,c}=0. This is analogous to the Magnetisation Transfer Insensitive Labeling Technique (TILT) used in spatially selective Pulsed Arterial Spin Labeling (PASL). In the TILT tag condition two +90° RF pulses are played out to produce a spatially selective 180° inversion, and in the control condition a +90°-90° RF pattern leaves the blood unperturbed but balances magnetisation transfer effects. Due to the conceptual similarity with TILT, albeit with the different goal of balancing diffusion effects, the novel velocity selective labeling scheme developed in the present invention is referred to as Velocity Selective TILT (VS-TILT).

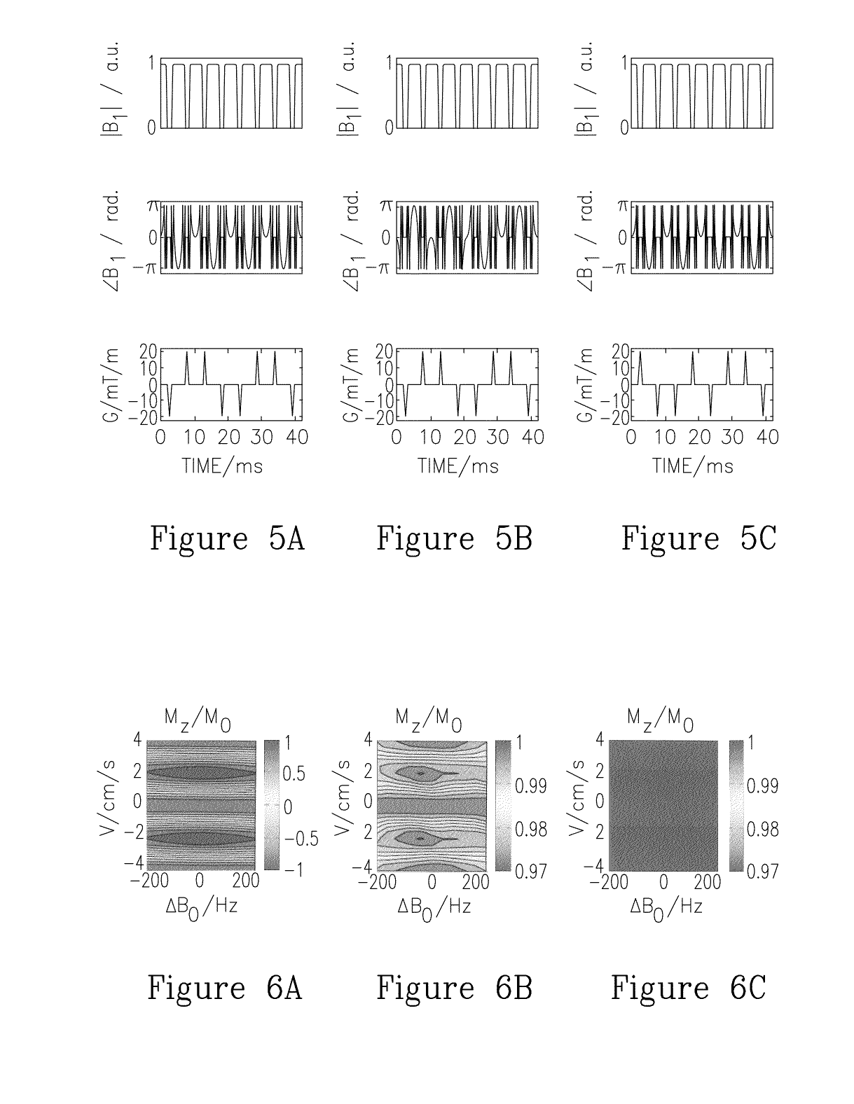

Any velocity selective pulse can be inserted for VS-A and VS-B, and there is also some freedom in how to negate the m₁ of either VS-A or VS-B in the control condition. Due to the robustness to B₁ inhomogeneity the BIR-4 (FIG. 4A) and BIR-8 (FIG. 5A) pulses are used as the base velocity selective pulse in the present invention. To negate the m₁ of VS-A for the control condition either the gradient polarity can be negated (FIGS. 4C and 5C), or the frequency sweep of the adiabatic pulse can be negated (FIGS. 4B and 5B). Neither method will alter the b-value of the composite pulse as this is proportional to the gradient squared

b=γ²∫₀^{TE}[∫₀^{t}G(t′)dt′]²dt (1.12)

whereas the m₁ linearly proportional to the applied gradient,

m₁=∫₀^{t}G(t′)t′dt′ (1.13)

Since it is possible that negating the gradient polarities for the control condition may be sensitive to eddy current effects, this was simulated and tested experimentally. In the next section the four permutations of velocity selective pulse (BIR-4 or BIR-8) and m₁ negation method (gradient or frequency sweep negating) are simulated.

Bloch equation simulations were performed using MATLAB 2012a (The MathWorks Inc., Natick, Mass., USA). The simulation considers rotations of the normalised magnetisation about B_{eff} with a time step of 5 μs. For these simulations relaxation times were ignored and the magnetisation was initially fully relaxed in the direction.

For each simulation two VS-TILT pulses were used (BIR-4 and BIR-8), and for each pulse type three conditions were simulated: the tag condition with

$${m}_{1} = \frac{π}{γ {V}_{cut}}$$

the control condition by negating the frequency sweep of VS-A, and the control condition by negating the gradient polarities of VS-A.

For all simulations V_{cut}=2 cm/s, the maximum gradient strength was 20 mT/m with a rise time of 0.5 ms and the maximum |B₁| was 20 μT unless otherwise specified. The BIR-4 and BIR-8 pulses used parameters ζ=15, tan(κ)=60, and ωₘₐₓ=39.8 kHz. The BIR-8 pulse used the +1:−1:−1:+1 gradient pattern to minimise eddy current effects. Of course other cut off velocities are equally applicable, the value of 2 cm/s is merely one example.

The effect of B₀ inhomogeneity on the tagging efficiency was simulated with ΔB₀ offset from −250 Hz to +250 Hz in steps of 50 Hz and the velocity of the spin packet from −4 cm/s to 4 cm/s in steps of 0.4 cm/s. Again, other values can equally be used.

The effect of eddy currents on static spins was simulated by convolving the desired gradient waveform with an eddy current of amplitude 0.25%. The eddy current time constants, T, were logarithmically spaced from 10⁻⁴ s to 1 s in 9 steps, and the position of the static spins from gradient isocentre ranged from −24 cm to +24 cm in steps of 4 cm.

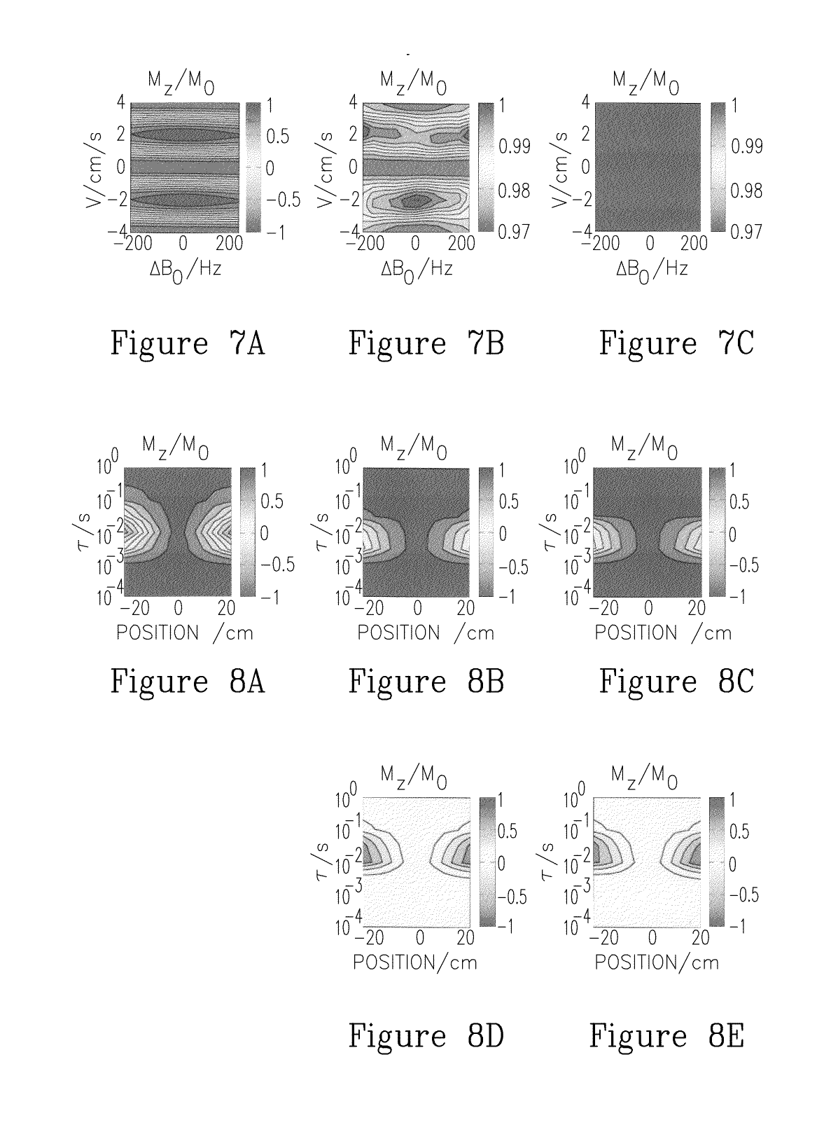

Displayed in FIGS. 6A to 6C and 7A to 7C are the final M_{z} for moving spins after the application of the BIR-4 VS-TILT preparations (FIGS. 6A to 6C) and BIR-8 VS-TILT preparation (FIGS. 7A to 7C). For both tag conditions the VS-TILT preparation imparts the desired cosinusoidal variation in velocity (FIGS. 6A and 7A). When ignoring relaxation the labeling efficiency of both BIR-4 VS-TILT and BIR-8 VS-TILT is reduced to 90% as off resonance is increased to 250 Hz.

After the application of the velocity selective control preparation M_{z}/M₀ is ideally 1 for all velocities. Both the BIR-4 and BIR-8 control conditions when negating the frequency sweep produce an inadequate control condition for moving spins (FIGS. 6B and 7B) in the presence of off-resonance. The minimum M_{z}/M₀ when negating the frequency sweep is 97.3% for BIR-4 VS-TILT and 97.0% for BIR-8 VS-TILT.

When negating the gradient polarities of VS-A for the control condition both the BIR-4 and BIR-8 produce more homogeneous control conditions (FIGS. 6C and 7C). In this case the minimum M_{z}/M₀ when negating the frequency sweep is 99.9% for both BIR-4 VS-TILT and BIR-8 VS-TILT.

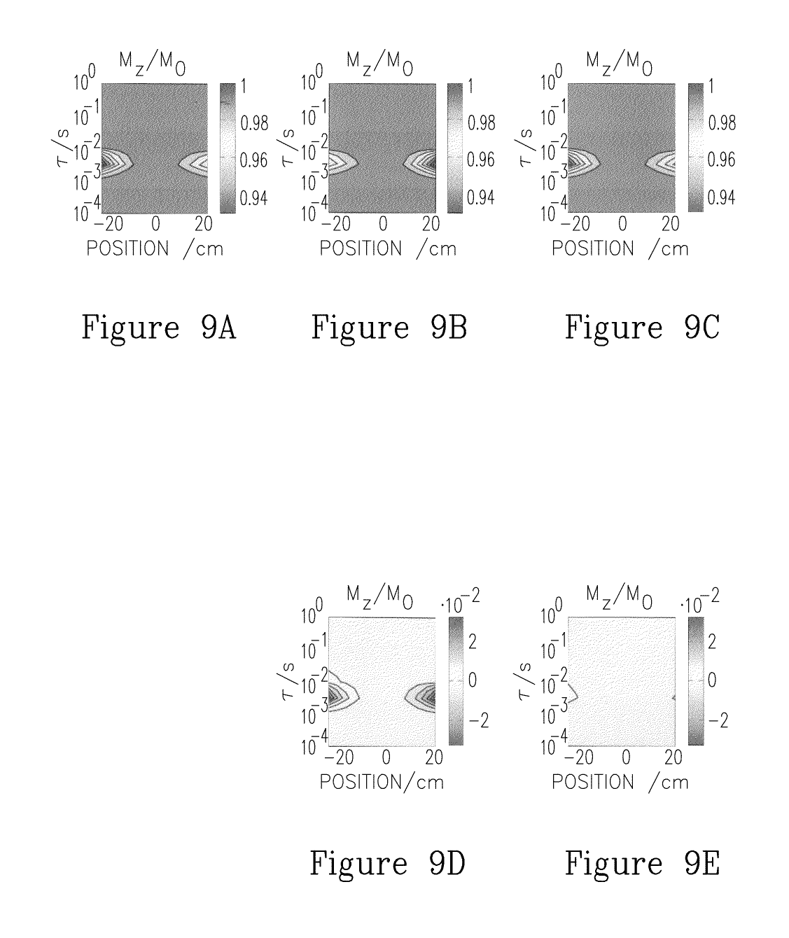

The final M_{z}/M₀ plots for static spins after the application of the VS-TILT preparation in the presence of eddy currents are displayed for BIR-4 VS-TILT (FIGS. 8A to 8E) and BIR-8 VS-TILT (FIGS. 9A to 9E). After the application of the preparation M_{z}/M₀ is ideally 1 for all static spins at all positions from isocentre. In the tag condition the BIR-4 VS-TILT is sensitive to eddy currents in the range 10−³ s to 10−¹ s, with minimum M_{z}/M₀=−66.0% (FIG. 8A). The eddy current sensitivity is reduced using BIR-8 VS-TILT, with minimum M_{z}/M₀=92.2% (FIG. 9A).

Eddy currents effects are not eliminated in the tag condition for either BIR-4 or BIR-8. Thus, in order that eddy currents do not produce artefacts, the control condition must produce the same eddy current spectrum such that the subtraction (|ΔM_{z}|/M₀) is 0. Both BIR-4 VS-TILT control conditions have a different eddy current response to the BIR-4 VS-TILT tag condition (FIGS. 8B and 8C, control condition negating frequency sweep and control condition negating gradients respectively). The |ΔM_{z}|/M₀ subtraction shows that the difference in the eddy current response would produce positive M_{z}/M₀ from static spins in the perfusion weighted image (FIG. 8D and FIG. 8E, when negating frequency sweep and negating gradients respectively). The overall effect is that the BIR-4 VS-TILT is most sensitive to eddy current time constants in the range 10⁻² s to 10⁻¹ s, with maximum |ΔM_{z}|/M₀96.0%.

For the BIR-8 VS-TILT control condition the reversed frequency sweep of VS-A produces an eddy current response that is spatially negated compared to the tag condition (FIG. 9B). The ΔM subtraction of this control condition indicates that eddy currents would produce both positive and negative artefacts (FIG. 9D), with maximum |ΔM_{z}|/M₀=3.7% for eddy current time constants on the order of 10⁻³ s. This error is reduced by using the BIR-8 VS-TILT gradient negation control condition (FIG. 9C), which has a similar eddy current response to the BIR-8 tag condition. In this case the maximum |ΔM_{z}|/M₀=0.6% (FIG. 9E).

The simulations suggest that the frequency sweep negation approach does not produce an adequate control condition for moving spins. This could be due to the switch from the negative frequency sweep to positive frequency sweep at the interface of VS-A and VS-B violating the adiabatic condition. The effect is not present when simulating the pulse at |B₁|=40 μT (data not shown), suggesting that the frequency sweep reversal control condition would have a higher adiabatic threshold than the 20 μT used in the simulations and the minimum |B₁|(r) that is achieved with body coil transmit.

The alternative to frequency sweep negation is to negate the gradient polarities for VS-A. The eddy current simulations suggest that both the BIR-4 VS-TILT control schemes will be more sensitive to eddy current effects than BIR-8 VS-TILT. The simulations also suggest that the BIR-8 VS-TILT with negated gradient polarities will be the least sensitive to eddy currents, and produce the optimal control condition for moving spins at the achievable |B₁|(r).

Following the simulations, the BIR-4 and BIR-8 VS-TILT preparations with gradient negation of VS-A were implemented in Siemens IDEA. Experiments were performed to determine the eddy current and diffusion errors by varying the V_{cut} and the labeling gradient axis in a static phantom. The tagging efficiency of BIR-8 VS-TILT was measured by varying the velocity of spins in a flow phantom using a single V_{cut} which was then compared to the BIR-8 VSASL preparation.

All experiments were performed on a 3 tesla Verio scanner (Siemens Healthcare, Erlangen, Germany) using a spin echo EPI readout. Of course, other instruments may also be used. Four preparations, BIR-4 and BIR-8 for both VSASL and VS-TILT labeling schemes, were evaluated for diffusion and eddy current effects. The subtraction errors were measured in a doped water phantom with ADC=2.7×10⁻³ mm²/s, T₁=100 ms and T₂=70 ms. To minimise any potential motion artefact the phantom was packed into the 32 channel receive coil using MR safe sandbags. For each preparation V_{cut}=1, 2, 4, 8 and 16 cm/s was used, and the experiments were repeated for each logical tagging gradient axis (\hat{x},ŷ and \hat{z}). A TR of 4 s allowed full relaxation of the spins prior to the application of the velocity selective pulse. Two dummy TRs were used followed by 4 tag/control pairs for each of the four preparations, five V_{cut} values and three labeling gradient directions.

The preparations used a maximum gradient strength of 20 mT/m, with the RF applied at the system maximum (nominal 24 μT). The preparations were applied TI=10 ms prior to acquisition of a single 5 mm axial slice. The readout incorporated crushers with VENC=V_{cut}, TE=28 ms with ⅞^{th} partial Fourier acquisition. The magnitude of the ΔM subtraction is then reported in a manually defined mask generated by thresholding an M₀ acquisition.

To evaluate the tagging efficiency the BIR-8 pulse was used in VS-TILT and VSASL labeling schemes and the response of spins in a flowing tube were measured. A peristaltic pump was set up in the control room to pump tap water through a 4 mm diameter tube which was fed through a waveguide in the RF screen. The tube was fed through the scanner bore, and looped around so that positive and negative velocities could be measured simultaneously. The two legs of the tube were taped to a board positioned so that the flow was along the \hat{z} direction.

A 10 mm coronal slice was prescribed with the readout in the \hat{z} direction. To avoid phase correction errors and minimise flow artefact a flyback spin echo EPI readout without crushers was used. The resolution in the phase encode direction was 6 mm to ensure that the voxel contained a laminar distribution of velocities. The two velocity selective preparations used V_{cut}=2 cm/s, maximum gradient strength of 20 mT/m and were applied TI=10 ms prior to the readout. A TR of 10 s was used along with a non-selective pre-saturation pulse to remove any spin history effects. Two dummy TRs were used followed by 4 tag/control pairs which were averaged.

The velocity of the spins in the tube was varied by adjusting a power index (υᵢ) on the pump from 0 to 10 in steps of 1 unit. The volume flow rate has previously been calibrated that relates the power index to Vₘₐₓ in cm/s by

Vₘₐₓ=(2.48±0.04)×υᵢ (1.14)

assuming laminar flow. However, the peristaltic pump has three rotating contact points with the tube and at power indices less than 5 some retrograde flow was observed due to inadequate contact. The data were therefore analysed without converting the flow to cm/s. For each power index the saturation efficiency, α(υᵢ) was calculated voxelwise as

$$α ( {v}_{i} ) = \frac{{m}^{\overline{c} trl} ( {v}_{i} ) - {m}^{\overline{t} ag} ( {v}_{i} )}{{M}_{0} ( {v}_{i} )} \qquad ( 1.15 )$$

where m ^{c̅trl}(υᵢ) is the average control magnetization, m ^{t̅ag}(υᵢ) is the average tag magnetization and M₀(υᵢ) is an acquisition at power index υᵢ without a velocity selective preparation applied. This was then averaged for each tube to get a measurement at +υᵢ, and −υᵢ. The mask was defined by a manual threshold at 10% signal of an M₀ acquisition.

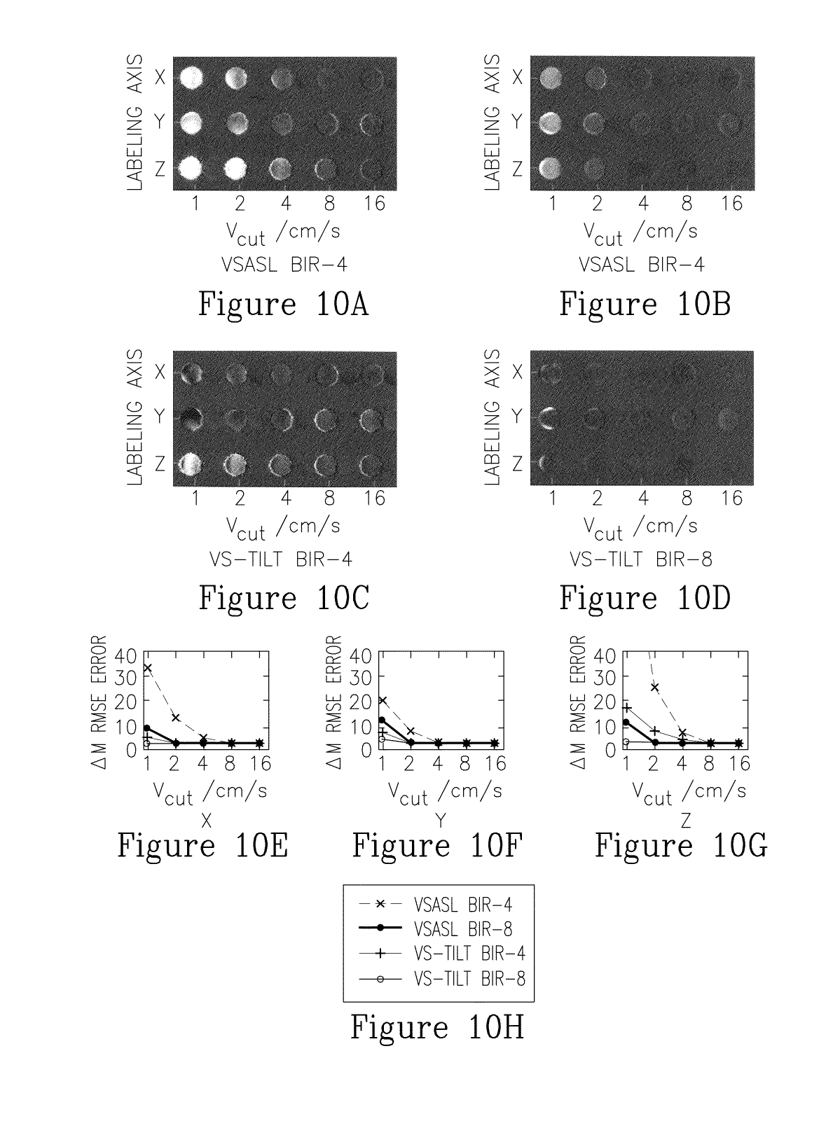

In the static phantom the VSASL BIR-4 produced the highest root mean squared subtraction error (RMSE) on all three axes (FIG. 10A). The error increased as V_{cut} was reduced due to the increase in both the diffusion b-value and eddy current effects. As shown above, the VSASL BIR-8 reduced the spatially dependent eddy current errors, but there is still residual signal due to diffusion (FIG. 10B). The RMSE values are reduced when using VS-TILT BIR-4 (FIG. 10C), but there is some negative signal when using X or Y labeling.

This residual signal is almost eliminated when using VS-TILT BIR-8 (FIG. 10D). However there are still some subtraction errors on the edges of the phantom at V_{cut}=1 cm/s. On all three labeling axes and for all V_{cut} the VS-TILT BIR-8 has the lowest RMSE value.

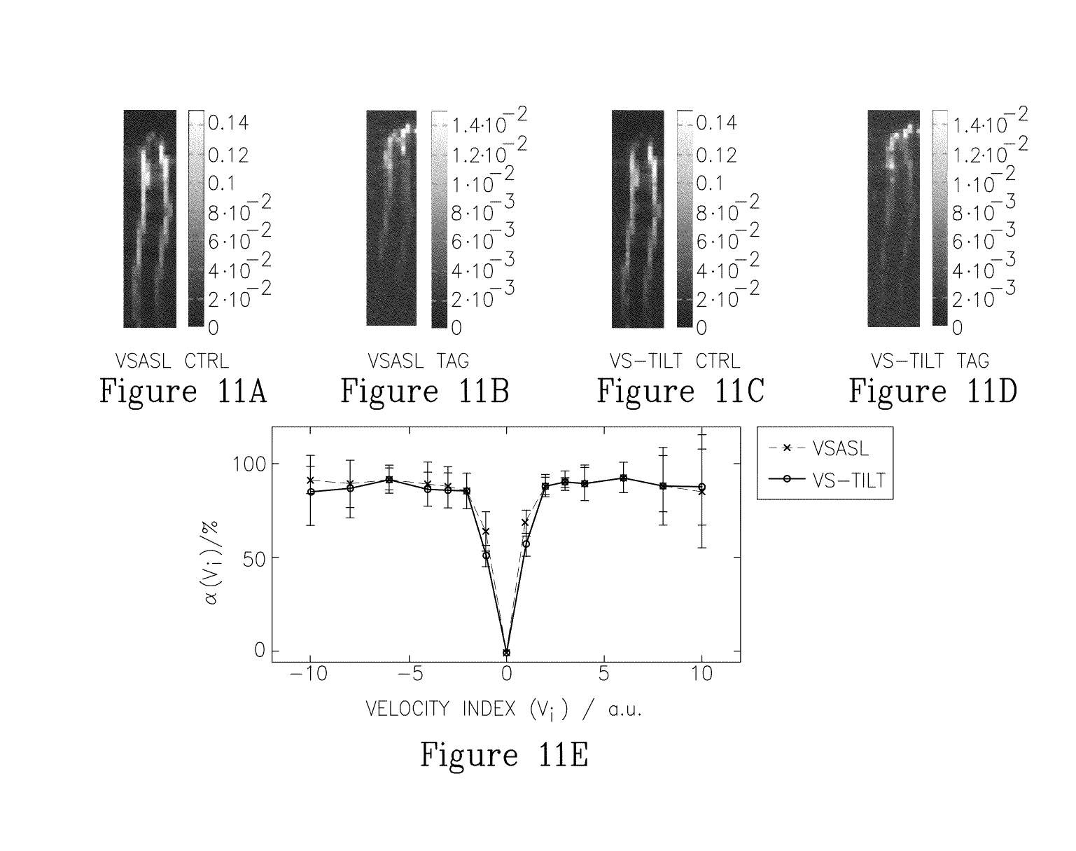

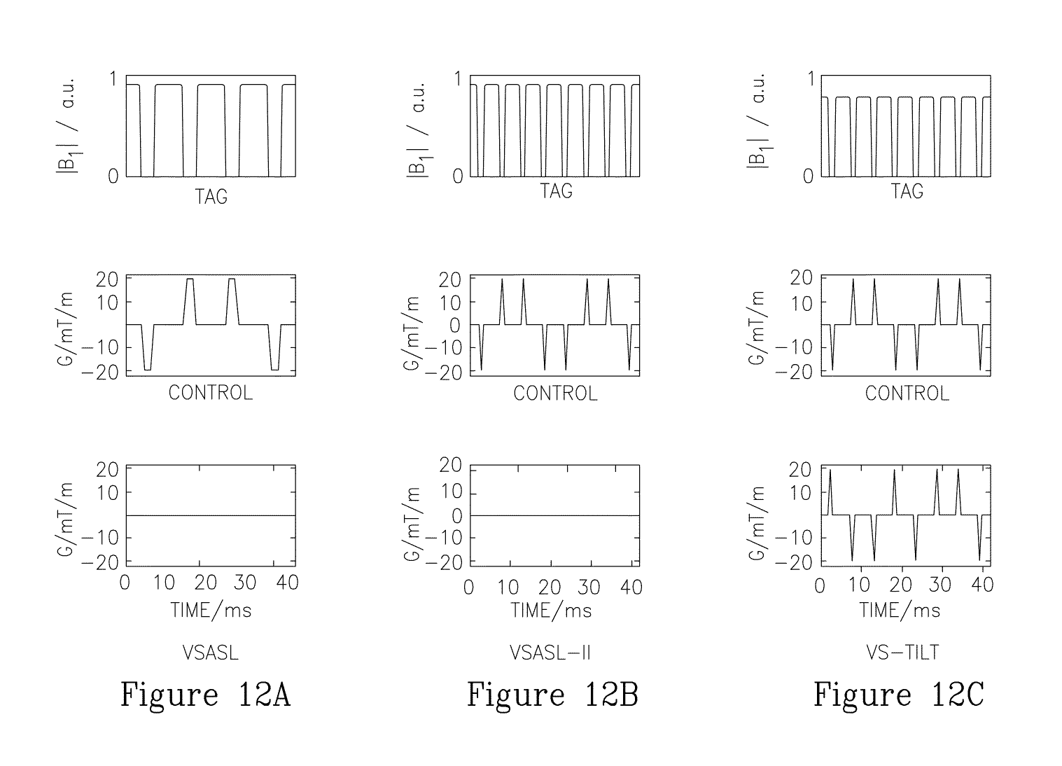

The BIR-8 VSASL and BIR-8 VS-TILT both produced saturations in flowing tap water. The saturation efficiency of both preparations was comparable (FIG. 11E). For |υᵢ|>2 the average efficiency was α=89.2±2.3% for VSASL and a=88.2±2.4% for VS-TILT.

The phantom experiments have shown that VS-TILT eliminates the diffusion error in a static phantom. Although the eddy currents are reduced when using VS-TILT compared to VSASL, the errors when using the BIR-4 pulse are larger than when using the BIR-8. However, the negative AM residual signal when using the BIR-4 was not predicted by the simulations. Potential reasons for this are an overcompensated eddy current term, or table vibration. Nevertheless, due to the reduced error in the static phantom for all V_{cut} the BIR-8 was used below.

The BIR-8 VSASL and BIR-8 VS-TILT were then tested in a flow phantom and found to produce similar saturation efficiencies. Due to the observed unstable flow at low velocity indices, and the lack of a phase contrast velocity measurement, the precise V_{cut} was not be determined. A rotating phantom may be more accurate. However, the absolute velocity of the spins in the flow phantom is not important. The key finding is that the BIR-8 VSASL and BIR-8 VS-TILT produce comparable labeling and control of flowing spins at a nominal V_{cut}=2 cm/s. The efficiency of the pulses (≈89%) in tap water might not be the same in vivo, due to the shorter T₁ and T₂ of arterial blood.

To investigate the diffusion effects in VSASL the BIR-8 VS-TILT preparation was compared to BIR-8 VSASL in vivo. It has been shown that the perfusion overestimation measured by VSASL increases as V_{cut} is reduced from 2 cm/s to 1 cm/s. This may be caused by diffusion effects as the b-value difference of the VSASL preparation increases as V_{cut} is reduced. Three preparations were therefore implemented to determine at which V_{cut} diffusion effects become significant.

The three velocity selective preparations used herewith are plotted in FIG. 12, all of which use BIR-8 as the basic velocity selective pulse. The VSASL (FIG. 12A) and VS-TILT (FIG. 12C) preparations have previously been described above. As the VS-TILT preparation is almost double the duration of the VSASL preparation this could result in decreased labeling efficiency. To match the labeling efficiency exactly, a third preparation is used, named VSASL-II. The VSASL-II preparation uses the VS-TILT tag preparation, but for the control acquisition, the velocity encoding gradients are turned off (FIG. 12B). The VSASL-II and VS-TILT will therefore have the same labeling efficiency, and the VSASL-II and VSASL will have similar b-value differences between the tag and control acquisitions.

Four healthy volunteers (two female, aged 23 to 33) were scanned having provided written consent under an institutionally agreed technical development protocol. For each of the three preparations values of V_{cut}=1, 2, 4, 8, 16 and 32 cm/s were acquired. The maximum gradient strength used was 20 mT/m and the ramp time was fixed at 0.5 ms. The diffusion b-values for each tag preparation are summarised in table 2 below:

| | Diffusion | | |
|---|---|---|---|
| V_{cut}/ | b-value/s/mm² | | |
| cm/s | VSASL | VSASL-II | VS-TILT |
| 1 | 5.32 | 3.12 | 3.12 |
| 2 | 1.56 | 0.872 | 0.872 |
| 4 | 0.436 | 0.228 | 0.228 |
| 8 | 0.114 | 0.0569 | 0.0569 |
| 16 | 0.0285 | 0.0142 | 0.0142 |
| 32 | 0.00712 | 0.00356 | 0.00356 |

The scan parameters were optimised in a preliminary pilot study, with the slice thickness increased to 10 mm to increase SNR. A spin echo EPI readout was used with 16 tag control pairs for each preparation and V_{cut} combination. Other parameters were TI=1 s, TR=4 s, TE=36 ms and 7 slices which resulted in a Tₛₐₜ=2.4 s. A DIR sequence was acquired and thresholded for use as grey matter mask. An M₀ acquisition without crushing gradients was segmented to obtain the CSF reference value.

Data were corrected for motion by registering to the M₀ image. Perfusion was then quantified voxelwise using the standard model (eq. 1.6). The same labeling efficiency was assumed for each pulse, with α=0.89. A grey matter mask was created by applying a manual threshold to the DIR acquisition. Mean grey matter perfusion values were calculated for each subject and preparation/V_{cut} combination by averaging over the grey matter mask. The grey matter perfusion values were then averaged over all subjects. Differences between the mean group perfusion for each preparation/V_{cut} combination were then evaluated using one-way ANOVA (Analysis of Variance).

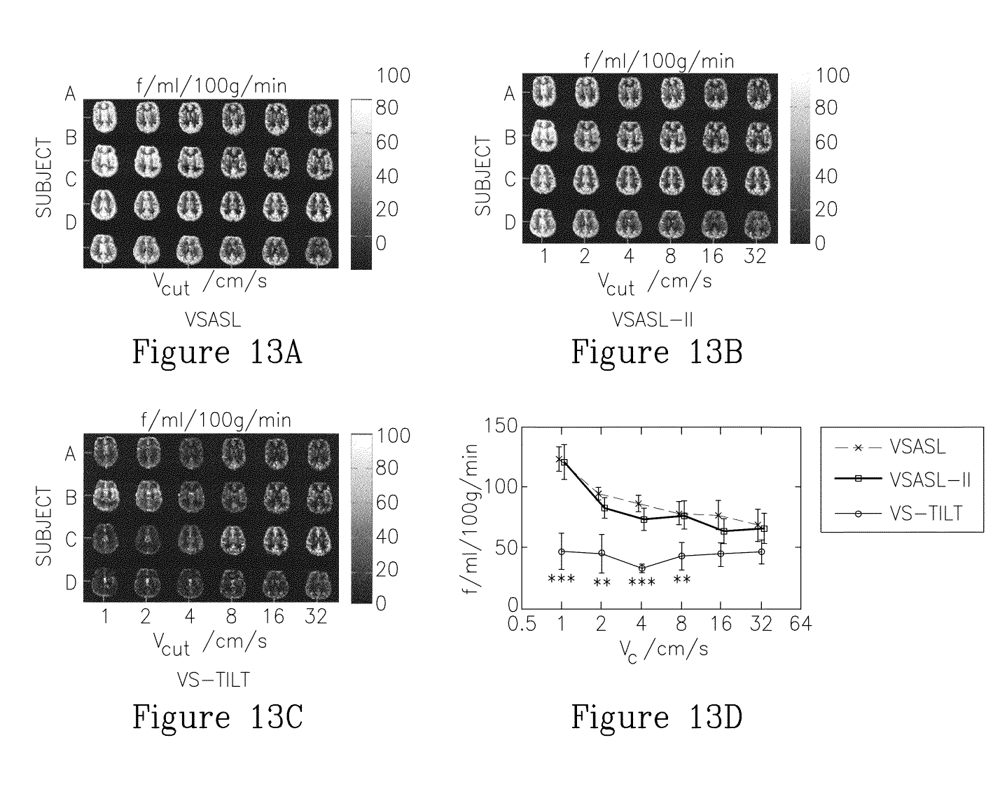

The quantified perfusion maps for a single slice containing the ventricles are displayed for VSASL (FIG. 13A), VSASL-II (FIG. 13B) and VS-TILT (FIG. 13C) for all subjects. It is observed that as V_{cut} is reduced there is increased signal in the ventricles for VSASL and VSASL-II. However, the grey-white matter contrast reduces when using VS-TILT.

The group mean perfusion for each preparation and V_{cut} is plotted in FIG. 13D, showing the mean grey matter f across all subjects. There are no significant differences between VSASL and VSASL-II at any V_{cut}. There are significant differences between VS-TILT and both VSASL and VSASL-H for V_{cut}=1 (P<0.001), 2 (P<0.01), 4 (P<0.001) and 8 (P<0.01) cm/s. At 2 cm/s, the mean perfusion measured by VSASL-II was 82.9±8.2 ml/100 g/min and VS-TILT was 44.9±16.1 ml/100 g/min.

The reduction in perfusion at the previously recommended V_{cut}=2 cm/s from 82.9±8.2 ml/100 g/min to 44.9±16.1 ml/100 g/min was not predicted by the simulations using physiological values for tissue ADC and relaxation time. Some potential reasons for this loss of signal include a reduction in labeling efficiency of VS-TILT, a reduction in the acceleration effects in VS-TILT, or that intravoxel incoherent motion could contribute significant signal to VSASL at V_{cut}=2 cm/s.

The labeling efficiency of VS-TILT is reduced compared to VSASL and was not corrected for in the quantification of the data. However, the labeling efficiency was exactly matched to the VS-TILT preparation by using the VSASL-II preparation. Although there was no significant difference between VSASL and VSASL-II perfusion measures, the values of apparent perfusion were consistently lower when using VSASL-II, suggesting there is a small difference in labeling efficiency. Although the VSASL-II and VS-TILT preparations are over 40 ms long the decay during the adiabatic pulse is not purely T₂, so the signal is preserved by T₁ decay. One problem with doubling the number of segments is that SAR is increased, although at TR=4 s the acquisition was not SAR limited.

Although the VS-TILT preparation is designed to be insensitive to diffusion, there is still some residual signal in the ventricles, that increases as V_{cut} is reduced.

In the prior art, it has been disclosed that the acceleration moment can contribute a significant signal in the brain. In the control acquisition for VSASL and VSASL-II the gradients are turned off, so the acceleration moment, m₂, is zero. For VS-TILT there will be a finite m₂ in the control. As with the first gradient moment, the variation in phase caused by m₂ will be tipped up to M_{z}, at the end of the velocity selective pulse. The difference in m₂ would therefore contribute positive signal in the ΔM subtraction. The values for m₁ and m₂ are summarized in table 3 below:

| | | VSASL | | VSASL-II | | VS-TILT | | |
|---|---|---|---|---|---|---|---|---|
| V_{cut}/ | m₁^{tag}/ | m₂^{tag}/ | m₂^{ctrl}/ | m₂^{tag}/ | m₂^{ctrl}/ | m₂^{tag}/ | m₂^{ctrl}/ | \|m₂^{ctrl}\| − \|m₂^{tag}\|/ |
| cm/s | s²T/m | s³T/m | s³T/m | s³T/m | s³T/m | s³T/m | s³T/m | s³T/m |
| 1 | 1.17 × 10⁻⁶ | 3.15 × 10⁻⁸ | 0 | 5.43 × 10⁻⁸ | 0 | 5.43 × 10⁻⁸ | −2.71 × 10⁻⁸ | 2.72 × 10⁻⁸ |
| 2 | 5.87 × 10⁻⁷ | 1.36 × 10⁻⁸ | 0 | 2.45 × 10⁻⁸ | 0 | 2.45 × 10⁻⁸ | −1.22 × 10⁻⁸ | 1.23 × 10⁻⁸ |
| 4 | 2.94 × 10⁻⁷ | 6.12 × 10⁻⁹ | 0 | 1.17 × 10⁻⁸ | 0 | 1.17 × 10⁻⁸ | −5.87 × 10⁻⁹ | 5.83 × 10⁻⁹ |
| 8 | 1.47 × 10⁻⁷ | 2.94 × 10⁻⁹ | 0 | 5.87 × 10⁻⁹ | 0 | 5.87 × 10⁻⁹ | −2.94 × 10⁻⁹ | 2.93 × 10⁻⁹ |
| 16 | 7.34 × 10⁻⁸ | 1.47 × 10⁻⁹ | 0 | 2.94 × 10⁻⁹ | 0 | 2.94 × 10⁻⁹ | −1.47 × 10⁻⁹ | 1.47 × 10⁻⁹ |
| 32 | 3.67 × 10⁻⁸ | 7.34 × 10⁻¹⁰ | 0 | 1.47 × 10⁻⁹ | 0 | 1.47 × 10⁻⁹ | −7.34 × 10⁻¹⁰ | 7.36 × 10⁻¹⁰ |

The calculated values suggest that the m₂ difference in VSASL-II is almost twice that of VSASL. However, the signal in VSASL-II is consistently lower than VSASL. For VS-TILT, the magnitude subtraction of the m₂, taken as m₂ is projected on to the \hat{z} axis, suggests that the acceleration contribution will be similar between VSASL and VS-TILT. In addition to this, m₂ is two orders of magnitude lower than m₁ so is an unlikely source of the difference between VS-TILT and VSASL-II.

The difference between VS-TILT and VSASL-II is potentially explained by the increased ADC measured at b-values<200 s/mm² in Intravoxel Incoherent Motion (IVIM) experiments as described in the prior art. In that prior art, it is hypothesised that incoherent flow produces a distribution of phase within a voxel that result in a reduction of magnitude of the signal. This is the same process as diffusion, but on a larger scale. Consequently, two apparent diffusion coefficients are fitted with a bi-exponential model, one that corresponds to fast diffusion (ADC*), another to slow diffusion (ADC). These are attributed to ‘perfusion’ and ‘tissue’ respectively. However, the fact that the data are well described by a bi-exponential model does not mean that there are two compartments in the data. For free water at 37° C. the ADC is 3×10−³ mm²/s, where as the measured ADC* in the brain is on the order of 7×10⁻³ mm²/s. To investigate this an IVIM experiment was performed to determine the ADC of tissue in the grey matter mask used in VSASL.

The increased ADC when using b-values<200 s/mm² has been previously observed. However, in the human brain, IVIM techniques have not been validated as a measure of perfusion, and it has been argued that IVIM cannot provide a measure of classical perfusion as it is sensitive to all incoherent flow in the voxel. In liver tumours, IVIM has been shown to correlate with histological staining for necrosis, not viable tissue. In animal models the fast component correlated with interstitial fluid pressure. It has also been shown that the IVIM fast component is reduced when using a FLAIR pulse, suggesting that the origin in the healthy brain is from. In VSASL, the aim is to report on classical perfusion, or the rate of delivery of blood from the arterial system to the capillary bed. If the VSASL signal is contaminated from IVIM effects, which do not report only on classical perfusion, then the true perfusion will be overestimated.

In the following, a multiple b-value experiment was performed to measure the ADC of the tissue used in the grey matter masks in VSASL experiments. To determine if the fast compartment of IVIM reported on the motion of blood the experiment was repeated with a FLAIR preparation to exclude the CSF compartment and a VASO preparation to exclude the (arterial) blood compartment.

The work was performed in one healthy volunteer (33 M) who had taken part in the previous VS-TILT study. Written consent was provided under an institutionally agreed technical development protocol. A single axial 10 mm slice was prescribed through the ventricles. Monopolar Stejkal-Tanner diffusion encoding gradients were applied in the \hat{z} direction with b-values 0 s/mm² to 100 s/mm² in steps of 10 s/mm2; 100 s/mm² to 200 s/mm² in steps of 20 s/mm²; 200 s/mm² to 500 s/mm² in steps of 50 s/mm²; and 500 s/mm² to 1000 s/rnm² in steps of 100 s/mm². The acquisition was a spin echo EPI with 96×96 matrix, 230 mm FOV, partial fourier 6/8^{th}, TE 80 ms to account for the time needed to achieve high b-values, TR 4 s. Four averages of each b-value were made. Following the readout was a non-selective saturation pulse to remove spin history effects. Of course, other parameters may be used.

The work was repeated with a VASO preparation, where a non-selective BASSI inversion was applied 1009.4 ms prior to the readout to null arterial blood species with T₁=1.664 s at the time of acquisition. For the FLAIR preparation, the non-selective BASSI inversion was applied 1483.9 ms prior to the excitation, to null CSF species with T₁=3.7 s. A DIR acquisition was also acquired for a grey matter mask.

The data were registered using 2D FLIRT. The grey matter mask was generated by applying a manual threshold to the DIR image, as in the VS-TILT work. For each b-value the data were averaged within the mask across all repetitions. For each preparation (IVIM, VASO-IVIM and FLAIR-IVIM) the data were first quantified to a mono-exponential model

S=S₀exp(−b S=S₀exp(−b·ADC) (1.16)

which was then used as a starting point for a non-linear least squares fit to a bi-exponential model using the MATLAB trust region reflective algorithm, bound to restrict f* to between 0 and 1:

S=S₀·([1−f*]·exp[−b·ADC_{B1}]+f*·exp[−b·ADC*]) (1.17)

where f* is the fast component fraction, ADC* is the fast diffusion coefficient and ADC_{B1} is the slow diffusion coefficient. To determine if the bi-exponential model is overfitting by introducing two new degrees of freedom, a Bayesian Information Criterion (BIC) was used, given by

$$BIC = n \cdot ln [ \frac{1}{n} {\sum }_{i = 1}^{n} {( {x}_{i} - {\hat{x}}_{i} )}^{2} ] + k \cdot ln ( n ) \qquad ( 1.18 )$$

where n is the number of data points, xᵢ are the data, \hat{x}ᵢ are the model's estimate of the data and k is the number of degrees of freedom in the model. The model with the lowest BIC is then selected, so the bi-exponential model is only chosen if the data support the extra degrees of freedom.

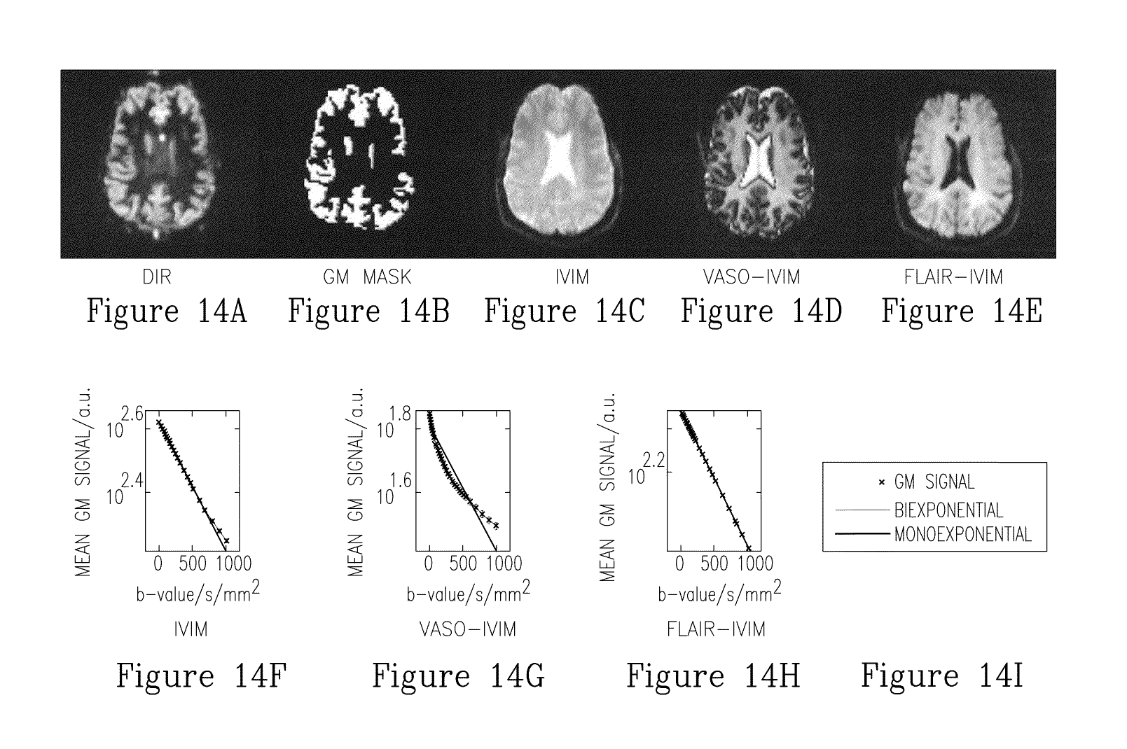

The acquired b=0 images are displayed in FIG. 14. The mask in FIG. 14B is generated by manually setting the threshold of the DIR image (FIG. 14A), which has been normalised for receive coil sensitivity. The data averaged over the mask for IVIM, VASO-IVIM and FLAIR-IVIM (FIG. 14C-E) are displayed in FIGS. 14F-H respectively. For IVIM and VASO-IVIM the BIC was lower for the bi-exponential mono-exponential model. However, for FLAIR-IVIM the BIC was lower for the mono-exponential fit.

The values of apparent diffusion coefficients are reported in table 4 below:

| | Mono | Bi-exponential | | | |
|---|---|---|---|---|---|
| | ADC/ | ADC_{BI}/ | ADC*/ | f*/ | BIC_{BI} < |
| Sequence | ×10⁻³ mm²/s | ×10⁻³ mm²/s | ×10⁻³ mm²/s | f₀ | BIC_{M} ? |
| IVIM | 0.92 ± 0.01 | 0.72 ± 0.01 | 4.6 ± 10.9 | 0.13 ± 0.01 | Yes |
| VASO-IVIM | 0.77 ± 0.02 | 0.38 ± 0.02 | 6.4 ± 11.8 | 0.27 ± 0.01 | Yes |
| FLAIR-IVIM | 0.68 ± 0.01 | (0.27 ± 0.82) | (0.87 ± 821.01) | (0.74 ± 0.01) | No |

For the fast diffusion coefficient, ADC*, a value of (4.6±10.9)×10−³ mm²/s was measured with IVIM and a value of (6.4+11.8)×10⁻³ mm²/s was measured with VASO-IVIM. The fractional signal size of the fast compartment, f*, was 0.13±0.01 and 0.27±0.01 respectively.

The voxelwise fits to the IVIM data were not significant, so a common approach is to average over the whole brain. The mask used in this work is the same as the one used in the VSASL data when taking an average over the perfusion values calculated voxelwise. The data show that a fast ADC is observed in the case of the IVIM work for low b-values, which is on the order of (4.6±10.9)×10⁻³ mm²/s. The elevated ADC also was observed when T₁ species of 1.664 ms (arterial blood) were nulled using a VASO pulse. However, the BIC indicated that that the data for FLAIR-I VIM was best described by a mono-exponential fit, so the fast component is not measurable when nulling T₁ species of 3700 ms. Note that the data point at b=0 for IVIM is an outlier for both models (FIG. 14F).

It is possible that the CSF or blood spins are not completely nulled by the BASSI pulse due to inefficiencies in the inversion, differences from the assumed T₁ or the delivered blood being outside the RF field of view when the magnetisation reset pulse is applied. In the VASO-IVIM data there may still be some capillary and venous blood present, as the T₁ is reduced as Oxygen is extracted. The size of each signal compartment in this work will not be the same as in VSASL, as a longer T₂ needs to be used in order to play out the diffusion encoding gradients in IVIM.

Nevertheless, these data show that for low b-values the ADC is elevated compared to prior art values of tissue diffusion. The hyper-ADC is observed even with a blood null pulse, but is absent when a FLAIR pulse is used. This suggests that the origin of the hyper-ADC is spins with long T₁, such as CSF. The elevated ADC is too large to be Brownian motion diffusion, but could be due to CSF flows in the subarachnoid spaces.

Here it has been shown that diffusion effects contribute significant positive signal to the ΔM subtraction for V_{cut}<8 cm/s. This was not predicted using literature values of tissue properties. However, at low b-values, the ADC is increased, even when the arterial blood signal is nulled. The likely source for this signal is from long T₁ species such as CSF. If a voxel of pure CSF had an ADC of 4.6×10−³ mm2/s, the error for TI=1 s is +54.5 ml/100 g/min for BIR-8 VSASL at V_{cut}, and for an ADC of 6.4×10−³ mm²/s the overestimation would be 74.5 ml/100 g/min. However, even with a partial volume of 20%, this would not completely explain the measured difference of 38 18 ml/100 g/min between VSASL-II and VS-TILT. It is possible that the ADC is even higher, as the first point in the IVIM is an outlier in the data. If only the b=0 s ADC is 53.8×10−³ mm²/s, which would describe the VS-TILT data.

Even using the conservative ADC*, there would still be significant contribution from the ‘fast’ spins in the VSASL measurement, which could be CSF. IVIM ADC* has been shown to report on tumour necrosis and interstitial fluid pressure, so it would be ideal to avoid these effects when interpreting data in the clinic. Although VS-TILT will have lower SNR than VSASL, the measurement will not be systematically biased from effects that are not related to classical perfusion.

The method of the present invention raises the question of what value of V_{cut} to use. There is still no adequate method of calibrating VSASL for this. As the effects of diffusion, motion and eddy currents are all increased as V_{cut} is reduced, there seems to be no benefit to reducing V_{cut} below 8 cm/s in the healthy brain. The choice of V_{cut}=2 cm/s has previously been argued by there being less evidence of “large vessel trees” and “grey matter is better depicted [at lower V_{cut} than higher V_{cut}]”. However, the reduction in artefacts may also be explained as being due to the fact that when the V_{cut} is reduced, the b-value difference is increased. This may introduce signal subtraction errors from the CSF in the voxel, acting as a blurring of the image and making the images appear less focal.

It has previously been assumed that the VSASL bolus is generated in a laminar flowing artery that is aligned with the gradient axis. Hereinafter, some of these assumptions are relaxed in order to determine the bolus shape in the presence of plug flow and under conditions of different distributions of arteries.

In VSASL it is assumed that the blood in the arteries is anisotropic coherent, in that the artery is aligned with the gradient axis and has a laminar distribution of velocities. It has been hypothesised that the VSASL tag cannot generate signal at the level of the capillaries due to the lack of laminar flow (Schmid et al., 2013). However, the shape of the labeled bolus will depend not only on the velocity profile within the vessel, but also the distribution of the vessels. In this derivation isotropic distributions of vessels are considered, where the orientations are uniformly distributed within the voxel.

The magnetisation profile after the application of a velocity selective pulse can be calculated from integrating over the distribution of vessels and their velocity profiles. The longitudinal magnetisation after a velocity selective pulse is given by

M_{z}(υ)=M₀α·cos[γm₁·υ·cos(θ)] (1.19)

where M₀ is the magnetisation prior to the velocity selective pulse, υ is the velocity of the spins and θ is the angle between the velocity and the direction of the applied gradient pulse. In VSASL the first moment of the gradients is chosen to be

$${m}_{1} = \frac{π}{γ {V}_{cut}} \qquad ( 1.20 )$$

which results in the longitudinal magnetisation after the application of a velocity selective pulse as:

$${M}_{z} ( v ) = {M}_{0} α \cdot cos [ π \frac{v}{{V}_{cut}} cos ( θ ) ] \qquad ( 1.21 )$$

A laminar vessel has a uniform distribution of velocities between 0 and υ_{MAX}. The normalised magnetisation within the vessel after the application of the pulse will therefore be given by the integral over the distribution of velocities present

$$\begin{matrix}{M}_{z}^{ANISO , LAM} = \frac{{\int }_{0}^{{v}_{MAX}} {M}_{z} ( v ) d v}{{\int }_{0}^{{v}_{MAX}} d v} \\ = \frac{{\int }_{0}^{{v}_{MAX}} {M}_{0} α \cdot cos [ π \frac{v}{{V}_{cut}} cos ( θ ) ] d v}{{\int }_{0}^{{v}_{MAX}} d v} ( 1.23 ) \\ = {M}_{0} α \cdot sin c [ π \frac{{v}_{MAX}}{{V}_{cut}} cos ( θ ) ] ( 1.24 )\end{matrix} \qquad ( 1.22 )$$

This sinc function is considered a saturation for laminar vessels aligned with the gradient axis (θ=0). If the vessel does not have laminar flow, but is instead plug flow with velocity υ_{MAX}, the normalised magnetisation after the application of the pulse is simply

$${M}_{z}^{ANISO , PLUG} = {M}_{0} α \cdot cos [ π \frac{ {v}_{MAX}}{{V}_{c ut}} cos ( θ ) ] \qquad ( 1.25 )$$

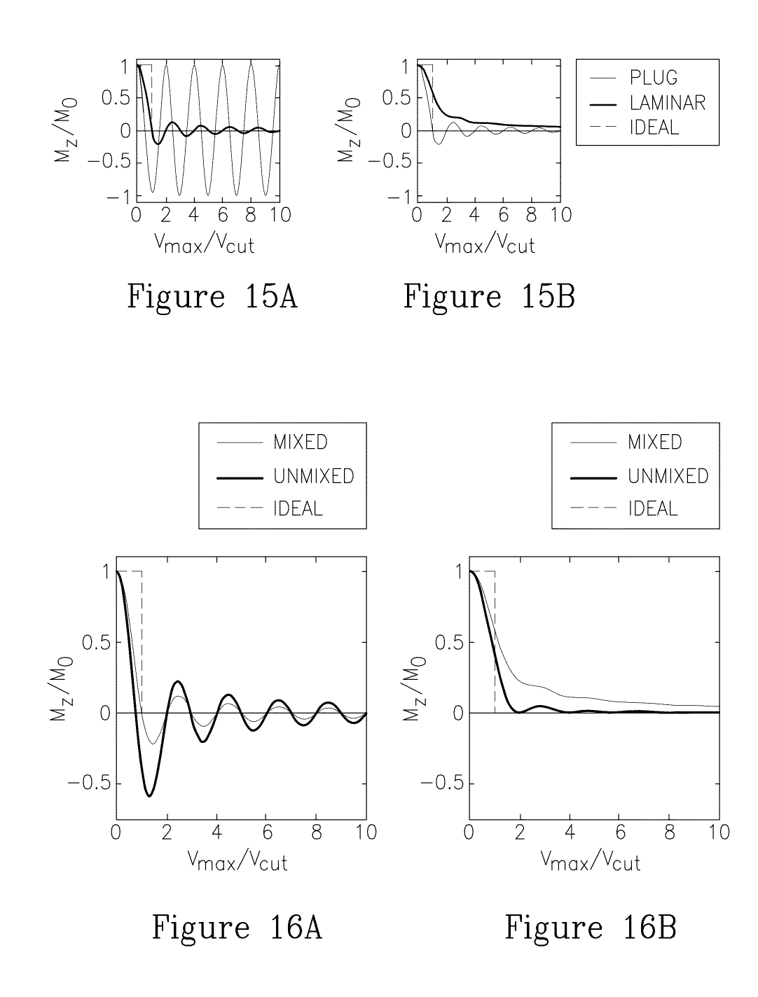

Whilst the cosine variation in M_{z}, will produce positive contrast in the ΔM subtraction, this cannot be considered a saturation so the tagging efficiency would be unclear. These bolus shapes for anisotropic laminar and plus vessels are plotted in FIG. 15a.

Consider a vessel that is not aligned with the velocity encoding axis. In this derivation spherical co-ordinates (r, θ, φ) with radial distance r, polar angle θ and azimuthal angle φ are used. For simplicity the tagging gradients are applied along the z axis. The bolus shape for an isotropic distribution of vessels is given by the integral over all the magnetisation in the individually distributed vessels

$${M}_{z}^{ISO} = \frac{\int \int {M}_{z} ( θ ) sin ( θ ) d θ d φ}{\int \int sin ( θ ) d θ d φ} \qquad ( 1.26 )$$

For an isotropic distribution of laminar vessels equation 1.24 can be substituted into 1.26 to yield:

$$\begin{matrix}{M}_{z}^{ISO , LAM} = \frac{\int \int {M}_{0} α \cdot sin c [ π \frac{{v}_{MAX}}{{V}_{cut}} cos ( θ ) ] sin ( θ ) d θ d φ}{\int \int sin ( θ ) d θ d φ} \\ = \frac{{M}_{0} α}{π \frac{{v}_{MAX}}{{V}_{cut}}} \cdot Si ( π \frac{{v}_{MAX}}{{V}_{cut}} ) ( 1.28 )\end{matrix} \qquad ( 1.27 )$$

where Si(x) is the sine integral defined by

$$Si ( r ) = {\int }_{0}^{z} \frac{sin ( {t}^{′} )}{{t}^{′}} d {t}^{′} \qquad ( 1.29 )$$

Due to rotational symmetry of the network this will hold for any tagging gradient direction. If an isotropic distribution of plug vessels is present the bolus shape will be given by substituting equation 1.25 into 1.26

$$\begin{matrix}{M}_{z}^{ISO , PLUG} = \frac{\int \int {M}_{0} α \cdot cos [ π \frac{{v}_{MAX}}{{V}_{cut}} cos ( θ ) ] sin ( θ ) d θ d φ}{\int \int sin ( θ ) d θ d φ} \\ = {M}_{0} α \cdot sin c ( π \frac{{v}_{MAX}}{{V}_{cut}} ) ( 1.31 )\end{matrix} \qquad ( 1.30 )$$

This result is interesting as an isotropic distribution of plug flowing vessels will produce a saturation. The bolus shape for the isotropic distribution of plug vessels is the same as a laminar vessel aligned along the labeling gradient axis.

The bolus shapes are summarised in table 5 below and plotted in FIG. 15, where FIG. 15A shows the anisotropic vessel distribution and FIG. 15B shows the isotropic vessel distribution.

| Vessel Velocity | Vessel Spatial Distribution | |
|---|---|---|
| Distribution | Anisotropic | Isotropic |
| Plug | ${M}_{O} α \cdot cos ( π \frac{{v}_{MAX}}{{V}_{cut}} cos ( θ ) )$ | ${M}_{O} α \cdot sin c ( π \frac{{v}_{MAX}}{{V}_{cut}} )$ |
| Laminar | ${M}_{B} α \cdot sin c ( π \frac{{v}_{MAX}}{{V}_{cut}} cos ( θ ) )$ | ${M}_{B} α \cdot \frac{S ( π \frac{{v}_{MAX}}{{V}_{cut}} )}{π \frac{{v}_{MAX}}{{V}_{cut}}}$ |

This derivation shows that when the vessel network is taken into account both plug flow and laminar flow produce a bolus that can be considered a saturation.

There is an apparent paradox with two assumptions of the labeling process in VSASL. Firstly, it is assumed that labeling will occur in a vessel with a laminar distribution of velocities. Secondly, after labeling the blood then mixes. The magnetisation delivered to the voxel is then proportional to

$$sin c ( \frac{π {v}_{max}}{{V}_{cut}} )$$

which is considered saturation for vessel velocities υₘₐₓ>υ_{cut}. The second assumption is incompatible with the first as in laminar flow there is no macroscopic mixing of fluid between layers. Instead, consider the magnetisation and volume of each velocity laminar that exit the vessel per unit time. Intuitively, a greater number of spins from the center of the vessel are going to be delivered as these have a higher velocity. The velocity of the laminar at radius r of the vessel is

$$v ( r ) = {v}_{max} ( 1 - \frac{{r}^{2}}{{R}^{2 }} ) \qquad ( 1.32 )$$

where R is the radius of the vessel. The volume flow rate from the vessel is given by

F=∫∫υ(r)dA=∫₀⁸υ(r)dr∫₀^{2x}dφ (1.33)

where φ is the azimuth angle within the vessel. The total magnetisation that exits the vessel per unit time is given by

$${M}_{z}^{ANSIO , UNMIXED} = \frac{\int \int {M}_{z} ( r ) v ( r ) d A}{F} = \frac{{\int }_{0}^{R} {M}_{z} ( r ) v ( r ) d r {\int }_{0}^{2 π} d φ}{{\int }_{0}^{R} v ( r ) d r {\int }_{0}^{2 π} d φ} \qquad ( 1.34 )$$

After the application of the velocity selective pulse the magnetisation of the laminar at r is

$${M}_{z} ( r , θ ) = cos [ π \frac{ {v}_{max}}{{V}_{ cut}} cos ( θ ) ( 1 - \frac{{r}^{2}}{{R}^{2}} ) ] \qquad ( 1.35 )$$

where θ is the angle between the vessel and the velocity selective gradient. Substituting equation 1.35 and equation 1.32 into equation 1.34 leads to the total magnetization delivered from a single laminar vessel

$${M}_{z}^{ANSIO , UNMIXED} = 2 \cdot sin c [ π \frac{{v}_{max}}{{V}_{cut}} cos ( θ ) ] - sin {c}^{2} [ π \frac{{v}_{max}}{2 {V}_{cut}} cos ( θ ) ] \qquad ( 1.36 )$$

This equation shows that at large υₘₐₓ/V_{cut} the sine term will dominate, but there will be some differences from the well-mixed case at low values of υₘₐₓ/V_{cut}. This function, along with the mixed bolus shape (eq. 1.24) is plotted in FIG. 16A (anisotropic vessel distribution). This can be extended to consider an isotropic distribution of vessels as in the previous section. Here the total magnetization delivered is given by

$${M}_{z}^{ISO , UNMIXED} = \frac{\int \int \int {M}_{z} ( r , θ ) v ( r ) d S}{F} = \frac{{\int }_{0}^{π} {\int }_{0}^{R} {M}_{z} ( r , θ ) v ( r ) sin ( θ ) d r d θ {\int }_{0}^{2 π} d φ}{{\int }_{0}^{R} v ( r ) {\int }_{0}^{2 π} d φ {\int }_{0}^{π} sin ( θ ) d θ} \qquad ( 1.37 )$$

which after a substitution and some algebra leads to

$${M}_{z}^{ISO , UNMIXED} = sin {c}^{2} ( π \frac{{v}_{max}}{2 {V}_{cut}} ) \qquad ( 1.38 )$$

This elegant solution suggests that an isotropic distribution of laminar vessels where the magnetization does not mix will result in the most efficient saturation. However, the first point at which this function is zero is when υₘₐₓ=2V_{cut}, which is twice the assumed cutoff velocity with an anisotropic laminar vessel. This function, along with the mixed bolus shape (eq. 1.28) is plotted in FIG. 16B (isotropic vessel distribution).

It is concluded from this derivation that even if the magnetization within the vessel does not mix, the total magnetisation that leaves the vessel will result in a saturation. In the case of an anisotropic laminar vessel the saturation efficiency would be higher if the spins mix. However, in the case of an isotropic network of laminar vessels the saturation efficiency is higher if the spins do not mix. In realistic blood flows the Reynolds Number is between 1 in arterioles to 4000 at peak systole in the aorta. Therefore, some mixing and flattening of the velocity profile may occur, so the true solution will be somewhere between the two extremes derived in this section that both result in saturation when the distribution of vessels is taken into account.

Although VS-TILT will exclude vascular networks that have incoherent flows, the technique is still theoretically generating a saturation in the capillary and arterioles.

This derivation suggests that untangling the vascular distribution from the velocity distribution is difficult in VSASL. However, it is possible to apply a velocity selective pulse that results in 114⁻, a sin(v) by adding phase to alternate AHPs in the BIR preparations. In this case, opposed velocities in an isotropic distribution of vessels will cancel out, leaving only the contributions from anisotropic vessels along the direction of the velocity selective gradient. By eliminating the isotropic contributions the technique will have lower SNR, but it could potentially be useful to study this geometry distribution during vascular normalisation in anti-angiogenic treatment.

In the method of the present invention, it has been shown that the magnitude of the perfusion error due to tissue diffusion and bulk motion effects can be been derived. A novel labeling scheme was then developed to eliminate diffusion effects, and it was shown that diffusion contributes an error of +38±18 ml/100 g/min in the grey matter masks for the conditions used in the simulation. Expressions for the bolus shape from non-laminar, non-isotropic vascular networks were then derived, demonstrating that a saturation is still produced when considering vascular networks for both plug and laminar flow. Finally, a paradox in two assumptions about the labeling process and delivery of blood from laminar vessels was then solved, and shown to produce the most efficient saturation in an isotropic vascular network.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A velocity selective preparation method, for Velocity Selective Magnetisation Transfer Insensitive labelling technique (VS-TILT), said VS-TILT method using non-selective RadioFrequency (RF) pulses and magnetic field gradients to modulate the longitudinal magnetization of moving spins in magnetic resonance imaging that is insensitive to diffusion effects, said method comprising the steps of:
a) play out two velocity selective pulses: VS-A and VS-B, sequentially without any spoiling between said pulses;
b) each individual pulse VS-A and VS-B having half the first gradient moment m₁ of the original velocity selective pulse;
c) assigning the VS-TILT tag condition gradients to have the same polarity, such that total m₁ is perserved;
d) assigning the VS_TILT control condition, negating the n gradients in the first pulse such total m₁=0, but the b-value remains unchanged.

2. The method of claim 1, wherein in the tag condition, two +90° RF pulses are played out to produce a spatially selective 180° inversion.

3. The method of claim 1, wherein in the control condition a +90°-90° pattern is played out, to balance magnetization transfer effects.

4. The method of claim 1, wherein a B₁ Insensitive Rotation pulse of order 4 is used as the base velocity selective pulse.

5. The method of claim 1, wherein a B₁ Insensitive Rotation pulse of order 8 is used as the base velocity selective pulse.

6. The method of claim 1, wherein a B₁ Insensitive Rotation pulse of order 16 is used as the base velocity selective pulse.

7. The method of claim 1, wherein a B₁ Insensitive Rotation pulse of order 32 is used as the base velocity selective pulse.

8. The method of claim 1, wherein the cut off velocity is set in the range 2-16 cm/s.
